(19) United States
(12) Patent Application Publication
(10) Pub. No.: US 2021/0027859 A1
(43) Pub. Date: Jan. 28, 2021

(54) Method, Apparatus and System to Detect Indels and Tandem Duplications Using Single Cell DNA Sequencing

(71) Applicant: Mission Bio, Inc., South San Francisco, CA (US)

(72) Inventors: Manimozhi Manivannan, South San Francisco, CA (US); Sombeet Sahu, South San Francisco, CA (US); Maurizio Pellegrino, San Francisco, CA (US)

(21) Appl. No.: 16/936,382

(22) Filed: Jul. 22, 2020

Related U.S. Application Data
(60) Provisional application No. 62/877,253, filed on Jul. 22, 2019.

(51) Int. Cl. G16B 30/00; G16B 20/20; G16B 20/50; G16B 35/00

(52) U.S. Cl. CPC G16B 30/00; G16B 35/00; G16B 20/50; G16B 20/20

(57) ABSTRACT

The disclosure generally relates to method, apparatus and system to detect indels and tandem duplications using single cell DNA sequencing. An exemplary method to detect one or more indel variants in a single cell DNA sequence may include the steps of: (1) obtaining a plurality of sequenced data sets from a cell sample having one or more indel variants, each of the plurality of sequenced data sets further includes a forward-direction sequencing read (R₁) and a reverse-direction sequencing read (R₂); (2) processing the plurality of sequenced data sets to identify a region of interest (ROI) in the forward-direction sequencing read (R₁) and in the reverse-direction sequencing read (R₂) for each of the plurality of sequenced data; (3) mapping each ROI to a known genome to identify target loci in each of R₁ and R₂ that do not map to the genome; (4) selecting a subset of the mapped ROIs with acceptable reads to identify a group of cells of interest; (5) from the selected subset, identifying one or more soft-clipped reads each ROI to identify a group of indel variants; and (6) determining at least one of location or frequency of occurrence for each indel variant of the identified group with respect to the corresponding ROI.

The instant disclosure claims priority to the Provisional Application No. 62/877,253, filed Jul. 22, 2019; the disclosure of which is incorporated herein in its entirety.

### SEQUENCE LISTING

The instant application contains a Sequence Listing which has been submitted electronically in ASCII format and is hereby incorporated by reference in its entirety. Said ASCII copy, created on Sep. 15, 2020, is named MSB-015US_SL.txt and is 806 bytes in size.

### FIELD

The instant disclosure generally relates to method, apparatus and system to detect indels and tandem duplications using single cell DNA sequencing. In an exemplary embodiment, the disclosure relates to detecting indels and tandem duplications in acute myeloid leukemia using single cell DNA sequencing.

### BACKGROUND

Assays are conventionally used for qualitatively assessing or quantitatively measuring the presence, amount, or functional activity of a target entity. The target entity, also known as the analyte, may be a DNA or an RNA fragment, a protein, a lipid or any other chemical compound whose presence can be detected. In some applications, assays have been developed to detect presence of a disease by detecting DNA/RNA sequences that correspond to the disease. For example, assays have been developed to detect the presence of multiple myeloma (MM) or acute myeloma (AM) in patients by detecting DNA fragments (or targets) that correspond to the disease. The timely and accurate detection of AM or MINI or other similar tumors is of significant interest to patients and the medical community.

Assay optimization and validation are essential, even when using assays that have been predesigned and commercially obtained. Optimization is implemented to ensure that the assay is as sensitive as is required. Assay optimization is also important to ensure that the assay is specific to the target of interest. For example, pathogen detection or expression profiling of rare mRNAs may require a high degree of sensitivity. Detecting a single nucleotide polymorphism (SNP) requires high specificity. On the other hand, viral quantification needs both high specificity and sensitivity.

Identification and removal of indels and tandem duplications in the final read are equally important as the assay optimization. Once the SNP is read, the data should be subject to further analysis and testing to identify an aberration or deletion where a specific nucleotide is present (i.e., insertion) or absent (i.e., deletion) in the raw data. Another common aberration is the presence of duplicate (e.g., tandem) SNP data in the raw data. Failure to identify such aberrations will result in the failure to detect the genome of interest or a false positive readout.

By way of example, FMS-like tyrosine kinase 3 receptor-internal tandem duplication (FLT3-ITD) commonly occurs in one-quarter of patients with acute myeloid leukemia. Acute leukemia has a poor prognosis, mainly due to relapse. Single-Cell DNA sequencing technologies, such as Tapestri® platform, allow a deeper understanding of the clonal heterogeneity of AML patient samples. Large indel calling is prone to errors from library preparation, sequencing biases, and algorithm artifacts. These errors contribute to false positives often in the form of multiple representations of the same variant.

There is a need to identify such aberrations with an algorithm and system to identify large indels in order to reduce false positives and to accurately measure the clonal heterogeneity for precision diagnostics.

### BRIEF DESCRIPTION OF THE DRAWINGS

The disclosed embodiments are discussed with reference to the following exemplary and non-limiting illustrations, in which like elements are numbered similarly, and where:

FIG. 1A is a representation of a single-stranded DNA sequence of a target molecule (FIG. 1A discloses SEQ ID NO: 2);

FIG. 1B shows a representation of paired end sequencing of a DNA strand;

FIG. 2 illustrates a flow diagram of an exemplary embodiment for identifying ITDs;

FIG. 3 is a flow diagram showing some of exemplary steps that may be implemented for ITD detection steps of FIG. 2

FIG. 4 is an exemplary illustration of a process to identify frequency of ITD occurrence per read; and

FIG. 5 shows an exemplary system for implementing an embodiment of the disclosure.

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

A ‘barcode’ nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

A barcode sequence can additionally be incorporated into microfluidic beads to decorate the bead with identical sequence tags. Such tagged beads can be inserted into microfluidic droplets and via droplet PCR amplification, tag each target amplicon with the unique bead barcode. Such barcodes can be used to identify specific droplets upon a population of amplicons originated from. This scheme can be utilized when combining a microfluidic droplet containing single individual cell with another microfluidic droplet containing a tagged bead. Upon collection and combination of many microfluidic droplets, amplicon sequencing results allow for assignment of each product to unique microfluidic droplets. In a typical implementation, we use barcodes on the Mission Bio Tapestri™ beads to tag and then later identify each droplet's amplicon content. The use of barcodes is described in U.S. patent application Ser. No. 15/940,850 filed Mar. 29, 2018 by Abate, A. et al., entitled ‘Sequencing of Nucleic Acids via Barcoding in Discrete Entities’, incorporated by reference herein.

In some embodiments, it may be advantageous to introduce barcodes into discrete entities, e.g., microdroplets, on the surface of a bead, such as a solid polymer bead or a hydrogel bead. These beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-spit cycle.

A barcode may further comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas a nucleic acid barcode sequence is used to distinguish between populations or groups of molecules. In some embodiments, where both a UMI and a nucleic acid barcode sequence are utilized, the UMI is shorter in sequence length than the nucleic acid barcode sequence.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. a-thio-nucleotide 5′-triphosphates. For a review of nucleic acid chemistry, see: Shabarova, Z. and Bogdanov, A. Advanced Organic Chemistry of Nucleic Acids, VCH, New York, 1994.

Any nucleic acid amplification method may be utilized, such as a PCR-based assay, e.g., quantitative PCR (qPCR), or an isothermal amplification may be used to detect the presence of certain nucleic acids, e.g., genes, of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

The number of amplification/PCR primers that may be added to a microdroplet may vary. The number of amplification or PCR primers that may be added to a microdroplet may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

One or both primer of a primer set may also be attached or conjugated to an affinity reagent that may comprise anything that binds to a target molecule or moiety. Nonlimiting examples of affinity reagent include ligands, receptors, antibodies and binding fragments thereof, peptide, nucleic acid, and fusions of the preceding and other small molecule that specifically binds to a larger target molecule in order to identify, track, capture, or influence its activity. Affinity reagents may also be attached to solid supports, beads, discrete entities, or the like, and are still referenced as affinity reagents herein.

One or both primers of a primer set may comprise a barcode sequence described herein. In some embodiments, individual cells, for example, are isolated in discrete entities, e.g., droplets. These cells may be lysed and their nucleic acids barcoded. This process can be performed on a large number of single cells in discrete entities with unique barcode sequences enabling subsequent deconvolution of mixed sequence reads by barcode to obtain single cell information. This approach provides a way to group together nucleic acids originating from large numbers of single cells. Additionally, affinity reagents such as antibodies can be conjugated with nucleic acid labels, e.g., oligonucleotides including barcodes, which can be used to identify antibody type, e.g., the target specificity of an antibody. These reagents can then be used to bind to the proteins within or on cells, thereby associating the nucleic acids carried by the affinity reagents to the cells to which they are bound. These cells can then be processed through a barcoding workflow as described herein to attach barcodes to the nucleic acid labels on the affinity reagents. Techniques of library preparation, sequencing, and bioinformatics may then be used to group the sequences according to cell/discrete entity barcodes. Any suitable affinity reagent that can bind to or recognize a biological sample or portion or component thereof, such as a protein, a molecule, or complexes thereof, may be utilized in connection with these methods. The affinity reagents may be labeled with nucleic acid sequences that relates their identity, e.g., the target specificity of the antibodies, permitting their detection and quantitation using the barcoding and sequencing methods described herein. Exemplary affinity reagents can include, for example, antibodies, antibody fragments, Fabs, scFvs, peptides, drugs, etc. or combinations thereof. The affinity reagents, e.g., antibodies, can be expressed by one or more organisms or provided using a biological synthesis technique, such as phage, mRNA, or ribosome display. The affinity reagents may also be generated via chemical or biochemical means, such as by chemical linkage using N-Hydroxysuccinimide (NETS), click chemistry, or streptavidin-biotin interaction, for example. The oligo-affinity reagent conjugates can also be generated by attaching oligos to affinity reagents and hybridizing, ligating, and/or extending via polymerase, etc., additional oligos to the previously conjugated oligos. An advantage of affinity reagent labeling with nucleic acids is that it permits highly multiplexed analysis of biological samples. For example, large mixtures of antibodies or binding reagents recognizing a variety of targets in a sample can be mixed together, each labeled with its own nucleic acid sequence. This cocktail can then be reacted to the sample and subjected to a barcoding workflow as described herein to recover information about which reagents bound, their quantity, and how this varies among the different entities in the sample, such as among single cells. The above approach can be applied to a variety of molecular targets, including samples including one or more of cells, peptides, proteins, macromolecules, macromolecular complexes, etc. The sample can be subjected to conventional processing for analysis, such as fixation and permeabilization, aiding binding of the affinity reagents. To obtain highly accurate quantitation, the unique molecular identifier (UMI) techniques described herein can also be used so that affinity reagent molecules are counted accurately. This can be accomplished in a number of ways, including by synthesizing UMIs onto the labels attached to each affinity reagent before, during, or after conjugation, or by attaching the UMIs microfluidically when the reagents are used. Similar methods of generating the barcodes, for example, using combinatorial barcode techniques as applied to single cell sequencing and described herein, are applicable to the affinity reagent technique. These techniques enable the analysis of proteins and/or epitopes in a variety of biological samples to perform, for example, mapping of epitopes or post translational modifications in proteins and other entities or performing single cell proteomics. For example, using the methods described herein, it is possible to generate a library of labeled affinity reagents that detect an epitope in all proteins in the proteome of an organism, label those epitopes with the reagents, and apply the barcoding and sequencing techniques described herein to detect and accurately quantitate the labels associated with these epitopes.

Primers may contain primers for one or more nucleic acid of interest, e.g. one or more genes of interest. The number of primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. Primers and/or reagents may be added to a discrete entity, e.g., a microdroplet, in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the PCR primers may be added in a separate step from the addition of a lysing agent. In some embodiments, the discrete entity, e.g., a microdroplet, may be subjected to a dilution step and/or enzyme inactivation step prior to the addition of the PCR reagents. Exemplary embodiments of such methods are described in PCT Publication No. WO 2014/028378, the disclosure of which is incorporated by reference herein in its entirety and for all purposes.

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

In some implementations, solid supports, beads, and the like are coated with affinity reagents. Affinity reagents include, without limitation, antigens, antibodies or aptamers with specific binding affinity for a target molecule. The affinity reagents bind to one or more targets within the single cell entities. Affinity reagents are often detectably labeled (e.g., with a fluorophore). Affinity reagents are sometimes labeled with unique barcodes, oligonucleotide sequences, or UMI's.

In some implementations, a RT/PCR polymerase reaction and amplification reaction are performed, for example in the same reaction mixture, as an addition to the reaction mixture, or added to a portion of the reaction mixture.

In one particular implementation, a solid support contains a plurality of affinity reagents, each specific for a different target molecule but containing a common sequence to be used to identify the unique solid support. Affinity reagents that bind a specific target molecule are collectively labeled with the same oligonucleotide sequence such that affinity molecules with different binding affinities for different targets are labeled with different oligonucleotide sequences. In this way, target molecules within a single target entity are differentially labeled in these implements to determine which target entity they are from but contain a common sequence to identify them from the same solid support.

In another aspect, embodiments herein are directed at characterizing subtypes of cancerous and pre-cancerous cells at the single cell level. The methods provided herein can be used for not only characterization of these cells, but also as part of a treatment strategy based upon the subtype of cell. The methods provided herein are applicable to a wide variety of caners, including but not limited to the following: Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Astrocytomas, Atypical Teratoid/Rhabdoid Tumor, Childhood, Central Nervous System (Brain Cancer), Basal Cell Carcinoma, Bile Duct Cancer, Bladder Cancer. Childhood Bladder Cancer, Bone Cancer (includes Ewing Sarcoma and Osteosarcoma and Malignant Fibrous Histiocytoma), Brain Tumors, Breast Cancer, Childhood Breast Cancer, Bronchial Tumors, Burkitt Lymphoma (Non-Hodgkin Lymphoma, Carcinoid Tumor (Gastrointestinal), Childhood Carcinoid Tumors, Cardiac (Heart) Tumors, Central Nervous System tumors. Atypical Teratoid/Rhabdoid Tumor, Childhood (Brain Cancer), Embryonal Tumors, Childhood (Brain Cancer), Germ Cell Tumor (Childhood Brain Cancer), Primary CNS Lymphoma, Cervical Cancer, Childhood Cervical Cancer, Cholangiocarcinoma, Chordoma (Childhood), Chronic Lymphocytic Leukemia (CLL), Chronic Myelogenous Leukemia (CML), Chronic Myeloproliferative Neoplasms, Colorectal Cancer, Childhood Colorectal Cancer, Craniopharyngioma (Childhood Brain Cancer), Cutaneous T-Cell Lymphoma, Ductal Carcinoma In Situ (DCIS), Embryonal Tumors, (Childhood Brain CNS Cancers), Endometrial Cancer (Uterine Cancer), Ependymoma, Esophageal Cancer, Childhood Esophageal Cancer, Esthesioneuroblastoma (Head and Neck Cancer), Ewing Sarcoma (Bone Cancer), Extracranial Germ Cell Tumors, Extragonadal Germ Cell Tumors, Eye Cancer, Childhood Intraocular Melanoma, Intraocular Melanoma, Retinoblastoma, Fallopian Tube Cancer, Fibrous Histiocytoma of Bone (Malignant, and Osteosarcoma), Gallbladder Cancer, Gastric (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis (Langerhans Cell Cancer), Hodgkin Lymphoma, Hypopharyngeal Cancer (Head and Neck Cancer), Intraocular Melanoma, Childhood Intraocular Melanoma, Islet Cell Tumors,(Pancreatic Neuroendocrine Tumors), Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Melanoma (Intraocular Eye), Childhood Intraocular Melanoma, Merkel Cell Carcinoma (Skin Cancer), Mesothelioma, Childhood Mesothelioma, Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes—see Unusual Cancers of Childhood, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CIVIL), Myeloid Leukemia, (Acute AML), Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer (Lip and Oral Cavity Cancer and Oropharyngeal Cancer), Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer, Parathyroid Cancer, Penile Cancer, Pharyngeal Cancer, Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Stomach (Gastric) Cancer, Childhood Stomach, T-Cell Lymphoma, Testicular Cancer, Childhood Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter Kidney (Renal Cell Cancer), Ureter and Renal Pelvis (Transitional Cell Cancer Kidney Renal Cell Cancer), Urethral Cancer, Uterine Cancer (Endometrial), Uterine Sarcoma, Vaginal Cancer, Childhood Vaginal Cancer, Vascular Tumors (Soft Tissue Sarcoma), Vulvar Cancer, Wilms Tumor (and Other Childhood Kidney Tumors).

Embodiments of the invention may select target nucleic acid sequences for genes corresponding to oncogenesis, such as oncogenes, proto-oncogenes, and tumor suppressor genes. In some embodiments the analysis includes the characterization of mutations, copy number variations, and other genetic alterations associated with oncogenesis. Any known proto-oncogene, oncogene, tumor suppressor gene or gene sequence associated with oncogenesis may be a target nucleic acid that is studied and characterized alone or as part of a panel of target nucleic acid sequences. For examples, see Lodish H, Berk A, Zipursky SL, et al. Molecular Cell Biology. 4th edition. New York: W. H. Freeman; 2000. Section 24.2, Proto-Oncogenes and Tumor-Suppressor Genes. Available from: https://www.ncbi .nlm . nih. gov/books/NBK21662/, incorporated by reference herein.

As used herein, the term “panel” refers to a group of amplicons that target a specific genome of interest or target a specific loci of interest on a genome.

As used herein, the term “Indel” refers to insertion or deletion of bases in the genome of an organization. Indel are classified among small genetic variations, for example, measuring from 1 to 10,000 base pairs in length. Indels may include insertion or deletion events that may be separated by many years or events and may not be unrelated to each other. A “microindel” as used herein is defined as an indel that results in a net change of 1 to 50 nucleotides. Indels (whether insertion or deletion) can be used as genetic markers in natural populations. It has been established that genomic regions with multiple indels can also be used to identify species. An indel change

An indel change of a single base pair in the coding part of an mRNA may result in the so-called frameshift during mRNA translation that could lead to an premature stop codon in a different frame. Indels that are not multiples of 3 are uncommon in coding regions but relatively common in non-coding regions. There are approximately 192-280 frameshifting indels in each person. It has been reported that indels are likely to represent between 16% and 25% of all sequence polymorphisms in humans. Most known genomes, including humans, indel frequency tends to be markedly lower than that of single nucleotide polymorphisms (SNP), except near highly repetitive regions, including homopolymers and microsatellites.

As used herein, the terms “tandem repeat” or “tandem duplication” occurs in DNA when a pattern of one or more nucleotides is repeated and the repetitions are directly adjacent to each other. A minisatellite is a repetition of between 10 and 60 nucleotides. Those with fewer repeats are known as microsatellites or short tandem repeats. When only two nucleotides are repeated, it is called a dinucleotide repeat (for example, “ACACACAC”). When only three nucleotides are repeated, it is called a trinucleotide repeat (for example, “AGCAGCAGCAG” (SEQ ID NO: 1)). Such abnormalities in a genomic region can give rise to trinucleotide repeat disorders. If the repeat unit copy number is variable in the population being considered, it is called a variable number tandem repeat (VNTR). Tandem repeats may occur through different mechanisms. For example, slipped strand mispairing, (also known as replication slippage), is a mutation process which occurs during DNA replication. It may include denaturation and displacement of the DNA strands, resulting in mispairing of the complementary bases. Slipped strand mispairing is one explanation for the origin and evolution of repetitive DNA sequences. Tandem repeats may also be the results of computation or reading anomalies inherent in the sequencing and the “read” operations.

As used herein, the term “homozygous” is used in a gene that has two identical alleles present in both homologous chromosomes. The cell in question is called homozygote. Th term “heterozygous” as used herein refers to a diploid organism in which the cells include two different alleles (i.e., a wild-type allele and a mutant allele) of a gene. The cell or organism is called a heterozygote for the specific allele. Thus, heterozygosity refers to a specific genotype. Heterozygous genotypes are represented by a capital letter (representing the dominant/wild-type allele) and a lowercase letter (representing the recessive/mutant allele), such as “Rr” or “Ss”. Alternatively, a heterozygote for gene “R” is assumed to be “Rr”.

As used herein, the term “circuitry” may refer to, be part of, or include an Application Specific Integrated Circuit (ASIC), an electronic circuit, a processor (shared, dedicated, or group), and/or memory (shared, dedicated, or group) that execute one or more software or firmware programs, a combinational logic circuit, and/or other suitable hardware components that provide the described functionality. In some embodiments, the circuitry may be implemented in, or functions associated with the circuitry may be implemented by, one or more software or firmware modules. In some embodiments, circuitry may include logic, at least partially operable in hardware. Embodiments described herein may be implemented into a system using any suitably configured hardware and/or software.

Other aspects of the disclosure are described in reference to the following exemplary embodiments and relate to method, system and apparatus to identify large indels and tandem variations in order to reduce false positive detections in genomic detections.

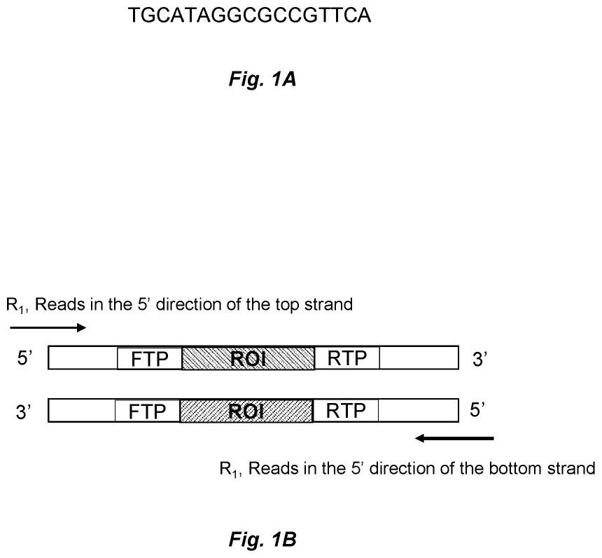

FIG. 1 is a representation of a single-stranded DNA sequence of a target molecule. Specifically, FIG. 1 illustrates a target DNA strand having 17 nucleotides. The target sequence of FIG. 1 may correspond to a mutation under study. Detection of the target DNA strand of FIG. 1, for example, may lead to detecting and identifying presence of sarcoma. To this end an assay may be designed and configured to specifically detect the presence of target DNA of FIG. 1.

FIG. 1B shows a representation of paired end sequencing of a DNA strand. Specifically, FIG. 1B shows two DNA strands side-by side. Each strand has a region of interest (ROI). The ROI is capped with a forward target primer (FTP) and a reverse target primer (RFP). Each strand is shown with a 3′ and a 5′ end. Finally, the read direction for both strand starts at the 5′ location and progresses toward the ROI as indicated by each of R₁ and R₂.

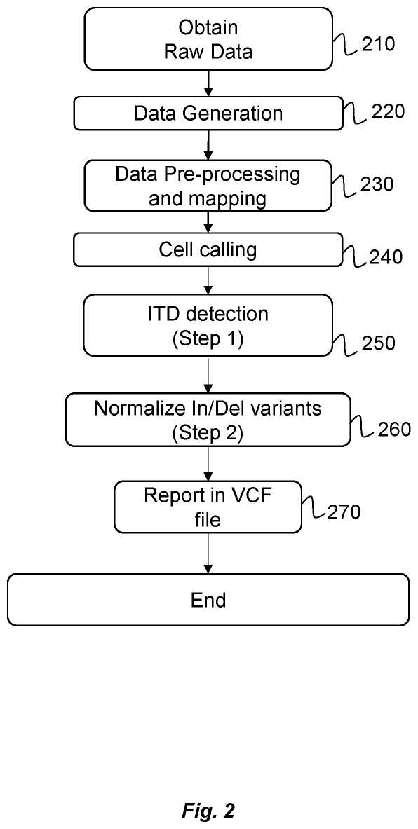

FIG. 2 illustrates an exemplary flow diagram of an exemplary embodiment. The Parts or all of the flow diagram may be implemented, for example, at software, hardware or a combination of software and hardware. In on embodiment, one or more apparatus may be used for implementing the steps of the flow diagram. To better illustrate the application of the disclosed embodiments, the implementation of this and other flow diagrams are provided below with reference to identification of aberration (Internal Tandem duplication or ITD) in the FLT3 gene. It should be noted that the disclosed principles are equally applicable to identifying aberrations in other genes and are not limited the exemplary embodiments provided herein.

At step 210, one or more experiments are run to obtain the primary raw data in order to identify the samples that are positive for ITD. The raw data may include bulk sequence data from one or more samples. The raw data may be analyzed with bulk sequencing to determine that the samples include ITD.

To further analyze this data, the raw from each sample may be processed through a sequencer to obtain an initial read of the Single Cell DNA (sDNA) corresponding to each sample. This is shown at step 220. Any conventional work flow may be used to prepare the sample for sequencing. In one example, the sequence length can in the range of about 150-20,000 amplicon base pairs (bps). In another example, the sequence length may be in the range of 200-2,000 bps. In still another example, the sequence length may be in the range of 25-200 bps. The sequence length may be adjusted and designed according to the specific application of the disclosed principles. The region of interest in each sample may also vary according to the application. For example, the region of interest of the sequenced sample may be in the range of about 20-50, 30-100, 100-500 or more than 500 bps. In an exemplary embodiment, the region of interest of the sequenced data may be about 220-270 bps.

Step 230 relates to data processing. Here, additional data processing steps are applied to the sequencing data in order to prepare the data for cell calling. Additional data processing steps may comprise, for example, barcode extraction, adaptor removal, mapping and removal of unmapped barcode regions. By way of example, the Burrows-Wheeler Alignment (BWA) technique may be applied to align (or map) the processed sequenced data to the human genome or to a sequence database. Step 230 may optionally include a filtering step to only keep sequence reads (hereinafter, reads) in which aberration is found.

In an exemplary embodiment, the results of steps 210-230 is stored in a so-called FASTQ file. A FASTQ file is a text file which contains the sequence data from the clusters that pass filter on a flow cell. The FASTQ file may be obtained from commercial sequencers, such as MiSeq® from Illumina® Corp. By way of example, for a single-read run, one Read 1 (R₁) FASTQ file may be created for each sample per flow cell lane. For a paired-end run, one R₁ and one Read 2 (R₂) FASTQ file may be created for each sample for each lane. The FASTQ files may be compressed and stored for additional data processing steps. Using conventional methods, regions of interest for each amplicon may be identified and stored.

Step 240 relates to cell calling. Cell calling may include one or more steps to identify complete cells from all the barcodes and to generate various plots and matrices of value. In one implementation, an amplicon cell-matrix is constructed in which the barcodes define the rows and the amplicons define the column of the matrix The value in each matrix box corresponds to the number of reads for that amplicon-barcode combination. TABLE 1 illustrates one such example:

TABLE 1
Exemplary Amplicon

| | BC 1 | BC 2 | BC 3 | . . . | BCn |
|---|---|---|---|---|---|
| Amp. 1 | Read 1, 1 | Read 1, 2 | Read 1, 3 | . . . | Read 1, n |
| Amp. 2 | Read 2, 1 | Read 2, 2 | Read 2, 3 | . . . | Read 2, n |
| Amp. 3 | Read 3, 1 | Read 3, 2 | Read 3, 3 | . . . | Read 3, n |
| . . . | | | | | |
| Amp. N | Read N, 1 | Read N, 2 | Read N, 3 | . . . | Read N, n |

In TABLE 1 each Read (R) may include data set of zero, one or multiple reads relating to the designated barcode and amplicon. Further each Read may include forward- and revere-direction reads (R₁, R₂). Next, a subset of the reads in the matrix are selected which contain at least one R. From this subset, a candidate list is selected in which each candidate has at least 8 times (8X) no of amplicon on the panel. That is, the subset identifies 80% of amplicons (and cells associated with those amplicons) that have good reads. This subset also identifies cells of interest.

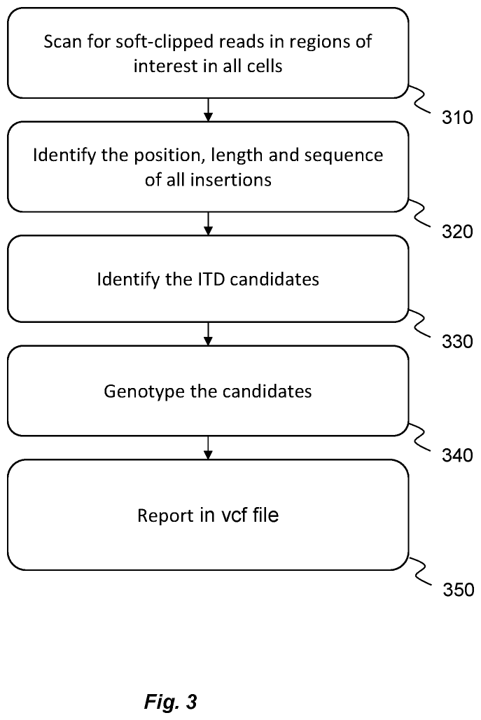

Step 250 is directed to aberration (e.g., ITD) detection. Here, the cells of interest which were identified at step 240 are further processed to identify cells with ITD. FIG. 3 is a flow-diagram for schematically showing some of the exemplary steps that may be implemented for ITD detection steps of FIG. 2.

Referring to FIG. 3, a step 310 the identified subset reads (step 240, FIG. 2) are scanned for soft-clipped reads in the regions of interest in all cells. There may be more than one ROI in each read. In an exemplary application, two regions of interest in each read is identified. The so-called soft-clipped reads are reads in which the sequence partially maps to the desired genome. For example, if two reads (R₁ and R₂) are obtained, a portion of R₁ and a portion of R₂ may map to the genome. A soft-clip may be due to an insertion event which would then cause the amplicon to be fully mapped into the genome.

At step 320, the positions, length and sequence of all soft-clipped insertion are identified and this data defines the subset of ITD candidates as shown in Step 330.

At step 340, the subset candidates are genotyped. In an exemplary implementation, if at least 20% of the read supports the ITD, the read is discarded as wildtype; if 20-90% of the read supports ITD, then the read is considered as heterozygous; and if more than 90% of the read supports ITD, then the read is considered as homozygous. Using this or similar criteria, at step 340, the reads are categorized based on the % of the read that supports ITD. This data is then stored at step 350. In an exemplary embodiment, the data is stored in Variant Call Format (VCF) file. The VCF file contains the results of the ITD detection step (Step 250, FIG. 2).

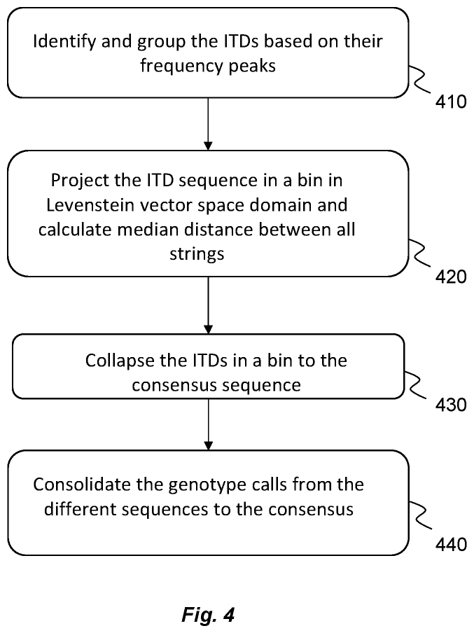

Reverting to FIG. 2, step 260 is directed to determining the frequency of ITD occurrence per base which leads to normalizing the insertion (In) or deletion (del) events. More specifically, this step determines where (in the Read) do ITD events occur and how frequently. While this determination may be implemented using different methodologies consistent with the disclosed principles, FIG. 4 shows one such exemplary method.

Referring to step 410 of FIG. 4, data from step 350 is reviewed to identify and group (bin) the ITDs based on their frequency peaks. The grouping can be made based on the location (or similarity of location within, for example, +/−20 bp of the location) where ITD occurs in each cell.

At step 420, the ITD sequence in a bin is projected in Levenshtein vector space domain and the median distances between all strings are calculated. That is, assuming that each bin contains the same variants of different lengths, collapse the entire bin into one string. Then using Levenshtein vector space domain, to calculate the median string distance which is considered ‘consensus’ of the sequence (See step 430). The consensus may be considered that correlates or corresponds to all of the sequences in the bin. This step allows grouping of all consensus variations into one sequence which enables breaking down a large volume of data into a manageable number of consensus sequences.

Referring again to FIG. 2, the genotype calls from the different consensus (step 430, FIG. 4) are consolidated and stored into the vcf file. The results collapse a large data set of ITD locations into a few consensus sequences in which the ITD location for each of the consensus sequences is known.

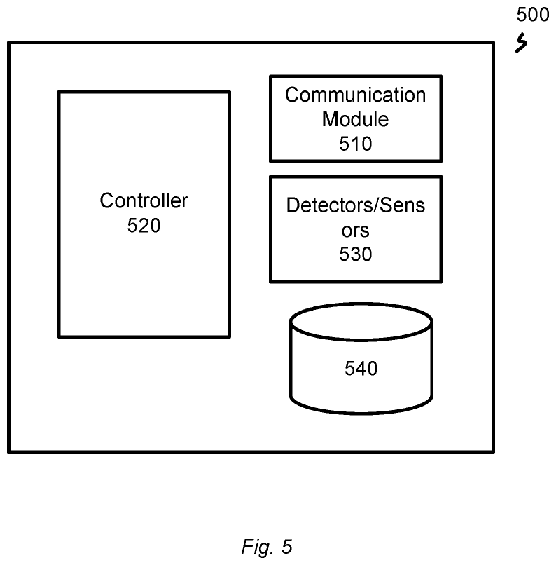

The flow-diagrams discussed in relation to FIGS. 2-4 may be implemented on software, hardware or a combination of software and hardware. FIG. 5 shows an exemplary system for implementing an embodiment of the disclosure. In FIG. 5, system 500 may comprise hardware, software or a combination of hardware and software programmed to implement steps disclosed herein, for example, the steps of flow diagram of FIG. 5. In one embodiment, system 500 may comprise an Artificial Intelligence (AI) CPU. For example, apparatus 500 may be an ML node, an MEC node or a DC node. In one exemplary embodiment, system 500 may be implemented at an Autonomous Driving (AD) vehicle. At another exemplary embodiment, system 500 may define an ML node executed external to the vehicle.

System 500 may comprise communication module 510. The communication module may comprise hardware and software configured for landline, wireless and optical communication. For example, communication module 510 may comprise components to conduct wireless communication, including WiFi, 5G, NFC, Bluetooth, Bluetooth Low Energy (BLE) and the like. Controller 520 (interchangeably, micromodule) may comprise processing circuitry required to implement one or more steps illustrates in FIGS. 2-4. Controller 520 may include one or more processor circuitries and memory circuities. Controller 520 may communicate with memory 540. Memory 540 may store one or more instructions to generate data tables, as described above, and to implement feature selection and statistical analysis, for example.

### EXAMPLE

The Tapestri® analytical workflow involves obtaining raw reads from the sequencer, removing adapters, aligning and mapping the reads, calling individual cells and identifying genetic variants within each cell.

In an exemplary application, we used a soft-clip based approach to detect the internal tandem duplications found in the FLT3 gene. The targeted panel had two amplicons targeting exons 14 and 15 in the FLT3 gene. The soft-clipped reads from these 2 amplicons were scanned for possible insertion events. The observed insertion event was qualified as an internal tandem duplication (ITD) variant if the total number of reads at the loci is greater than 10 and at least 20% of the reads support the insertion. The ITD variant was called homozygous if the allele frequency is greater than 0.9 and heterozygous otherwise.

We then applied a generalized median string in Levenshtein space to collapse the different indel variants. The generalized median string was defined as a string that had the smallest sum of distances to the elements of a given set of strings. To do this, we first identify the candidate ITD size bins from the frequency peaks of all the called ITD variants and group the individual variants that are within 20 bp boundaries of the frequency peaks into their respective bins. We projected the ITD sequence strings within a bin on to Levenshtein vector space domain and calculated the median distance between all strings. We then used the string with the median distance to collapse the ITDs to the consensus sequence and report it in the vcf file.

### Results

We processed AML samples with known FLT3 ITDs through Tapestri® platform. We analyzed the raw data via Tapestri® analytical workflow including large indel and ITD detection algorithm. Using this method, we were able to accurately identify the ITDs and reproduce the true positive clones for the sample. The disclosed principles may be applied to different samples with a wide range of known ITDs.

The disclosed embodiments are exemplary and non-limiting. It will be evident to one of ordinary skill in the art that the disclosed principles may be applied to different samples for similar identification without departing from the instant disclosure.

The following examples are provided to further illustrate the disclosed principles. These examples are non-limiting and illustrative. It is noted that one of ordinary skill in the art may modify the examples without departing from the disclosed principles.

Example 1 is directed to a method to detect one or more indel variants in a single cell DNA sequence, the method comprising: obtaining a plurality of sequenced data sets from a cell sample having one or more indel variants, each of the plurality of sequenced data sets further comprising a forward-direction sequencing read (R1) and a reverse-direction sequencing read (R2); processing the plurality of sequenced data sets to identify a region of interest (ROI) in the forward-direction sequencing read (R1) and in the reverse-direction sequencing read (R2) for each of the plurality of sequenced data; mapping each ROI to a known genome to identify target loci in each of R1 and R2 that do not map to the genome; selecting a subset of the mapped ROIs with acceptable reads to identify a group of cells of interest; from the selected subset, identifying one or more soft-clipped reads each ROI to identify a group of indel variants; and determining at least one of location or frequency of occurrence for each indel variant of the identified group with respect to the corresponding ROI.

Example 2 is directed to the method of example 1, wherein the indels comprises insertion and duplication events.

Example 3 is directed to the method of any previous example, wherein the cell sample comprises one ore more aberration.

Example 4 is directed to the method of any previous example, wherein the processing of the plurality of sequenced data further comprises removing at least one of a bar code or an adaptor from each of R1 and R2.

Example 5 is directed to the method of any previous example, wherein the mapping step further comprises removing an unmapped region of the sequenced data.

Example 6 is directed to the method of any previous example, wherein acceptable reads defines ROIs which conform to a genome of interest by at least 80%.

Example 7 is directed to the method of any previous example, wherein the identifying step further comprises at least one of length, position and sequence associated with a soft-clipped indel.

Example 8 is directed to the method of any previous example, wherein determining location of occurrence for each variant further comprises determining a location in the ROI where the indel occurs.

Example 9 is directed to the method of any previous example, wherein determining frequency of occurrence for each variant further comprises determining the frequency with which the indel variant occurs.

Example 10 is directed to the method of any previous example, wherein the step of determining at least one location or frequency of occurrence further comprises grouping similarly occurring indel variants and calculating, for each group, a consensus representative sequence.

Example 11 is directed to the method of any previous example, wherein the step of calculating a consensus representative sequence further comprises calculating a Levenshtein distance for each group of indel variants.

Example 12 is directed to a non-transient machine-readable medium including instructions to detect one or more indel variants in a single cell DNA sequence, which when executed on one or more processors, causes the one or more processors to: obtain a plurality of sequenced data sets from a cell sample having one or more indel variants, each of the plurality of sequenced data sets further comprising a forward-direction sequencing read (R1) and a reverse-direction sequencing read (R2); process the plurality of sequenced data sets to identify a region of interest (ROI) in the forward-direction sequencing read (R1) and in the reverse-direction sequencing read (R2) for each of the plurality of sequenced data; map each ROI to a known genome to identify target loci in each of R1 and R2 that do not map to the genome; select a subset of the mapped ROIs with acceptable reads to identify a group of cells of interest; from the selected subset, identify one or more soft-clipped reads each ROI to identify a group of indel variants; and determine at least one of location or frequency of occurrence for each indel variant of the identified group with respect to the corresponding ROI.

Example 13 is directed to the medium of example 12, wherein the indels comprises insertion and duplication events.

Example 14 is directed to the medium of examples 12-13, wherein the cell sample comprises one ore more aberration.

Example 15 is directed to the medium of examples 12-14, wherein the instructions to process the plurality of sequenced data further comprises removing at least one of a bar code or an adaptor from each of R1 and R2.

Example 16 is directed to the medium of examples 12-15, wherein the instruction to map each ROI further comprises removing an unmapped region of the sequenced data.

Example 17 is directed to the medium of examples 12-16, wherein acceptable reads defines

ROIs which conform to a genome of interest by at least 80%.

Example 18 is directed to the medium of examples 12-17, wherein the instruction to identify one or more soft-clipped reads further comprises identifying at least one of length, position and sequence associated with a soft-clipped indel.

Example 19 is directed to the medium of examples 12-18, wherein the instruction to determine location of occurrence for each variant further comprises determining a location in the ROI where the indel occurs.

Example 20 is directed to the medium of examples 12-19, wherein the instruction to determine frequency of occurrence for each variant further comprises determining the frequency with which the indel variant occurs.

Example 21 is directed to the medium of examples 12-20, wherein the instruction to determine at least one of location or frequency of occurrence further comprises grouping similarly occurring indel variants and calculating, for each group, a consensus representative sequence.

Example 22 is directed to the medium of examples 12-21, wherein calculating a consensus representative sequence further comprises calculating a Levenshtein distance for each group of indel variants.

What is claimed is:

1. A method to detect one or more indel variants in a single cell DNA sequence, the method comprising:
obtaining a plurality of sequenced data sets from a cell sample having one or more indel variants, each of the plurality of sequenced data sets further comprising a forward-direction sequencing read (R₁) and a reverse-direction sequencing read (R₂);
processing the plurality of sequenced data sets to identify a region of interest (ROI) in the forward-direction sequencing read (R₁) and in the reverse-direction sequencing read (R₂) for each of the plurality of sequenced data;
mapping each ROI to a known genome to identify target loci in each of R₁ and R₂ that do not map to the genome;
selecting a subset of the mapped ROIs with acceptable reads to identify a group of cells of interest;
from the selected subset, identifying one or more soft-clipped reads each ROI to identify a group of indel variants; and
determining at least one of location or frequency of occurrence for each indel variant of the identified group with respect to the corresponding ROI.

2. The method of claim 1, wherein the indels comprises insertion and duplication events.

3. The method of claim 1, wherein the cell sample comprises one ore more aberration.

4. The method of claim 1, wherein the processing of the plurality of sequenced data further comprises removing at least one of a bar code or an adaptor from each of R₁ and R₂.

5. The method of claim 1, wherein the mapping step further comprises removing an unmapped region of the sequenced data.

6. The method of claim 1, wherein acceptable reads defines ROIs which conform to a genome of interest by at least 80%.

7. The method of claim 6, wherein the identifying step further comprises at least one of length, position and sequence associated with a soft-clipped indel.

8. The method of claim 1, wherein determining location of occurrence for each variant further comprises determining a location in the ROI where the indel occurs.

9. The method of claim 1, wherein determining frequency of occurrence for each variant further comprises determining the frequency with which the indel variant occurs.

10. The method of claim 1, wherein the step of determining at least one location or frequency of occurrence further comprises grouping similarly occurring indel variants and calculating, for each group, a consensus representative sequence.

11. The method of claim A9, wherein the step of calculating a consensus representative sequence further comprises calculating a Levenshtein distance for each group of indel variants.

12. A non-transient machine-readable medium including instructions to detect one or more indel variants in a single cell DNA sequence, which when executed on one or more processors, causes the one or more processors to:
obtain a plurality of sequenced data sets from a cell sample having one or more indel variants, each of the plurality of sequenced data sets further comprising a forward-direction sequencing read (R₁) and a reverse-direction sequencing read (R₂);
process the plurality of sequenced data sets to identify a region of interest (ROI) in the forward-direction sequencing read (R₁) and in the reverse-direction sequencing read (R₂) for each of the plurality of sequenced data;
map each ROI to a known genome to identify target loci in each of R₁ and R₂ that do not map to the genome;
select a subset of the mapped ROIs with acceptable reads to identify a group of cells of interest;
from the selected subset, identify one or more soft-clipped reads each ROI to identify a group of indel variants; and
determine at least one of location or frequency of occurrence for each indel variant of the identified group with respect to the corresponding ROI.

13. The medium of claim 12, wherein the indels comprises insertion and duplication events.

14. The medium of claim 12, wherein the cell sample comprises one ore more aberration.

15. The medium of claim 12, wherein the instructions to process the plurality of sequenced data further comprises removing at least one of a bar code or an adaptor from each of R₁ and R₂.

16. The medium of claim 12, wherein the instruction to map each ROI further comprises removing an unmapped region of the sequenced data.

17. The medium of claim 12, wherein acceptable reads defines ROIs which conform to a genome of interest by at least 80%.

18. The medium of claim 17, wherein the instruction to identify one or more soft-clipped reads further comprises identifying at least one of length, position and sequence associated with a soft-clipped indel.

19. The medium of claim 12, wherein the instruction to determine location of occurrence for each variant further comprises determining a location in the ROI where the indel occurs.

20. The medium of claim 12, wherein the instruction to determine frequency of occurrence for each variant further comprises determining the frequency with which the indel variant occurs.

21. The medium of claim 12, wherein the instruction to determine at least one of location or frequency of occurrence further comprises grouping similarly occurring indel variants and calculating, for each group, a consensus representative sequence.

22. The medium of claim 21, wherein calculating a consensus representative sequence further comprises calculating a Levenshtein distance for each group of indel variants.


SEQUENCE LISTING (2 sequences)

SEQ ID NO: 1 - DNA, 11 bp, Artificial Sequence
  Description of Artificial Sequence Synthetic oligonucleotide
agcagcagcag

SEQ ID NO: 2 - DNA, 17 bp, Artificial Sequence
  Description of Artificial Sequence Synthetic oligonucleotide
tgcataggcgccgttca